(19) United States
(12) Patent Application Publication
(10) Pub. No.: US 2015/0045890 A1
(43) Pub. Date: Feb. 12, 2015

(54) INTERVERTEBRAL DISC PROSTHESIS MADE FROM THERMOPLASTIC MATERIAL HAVING GRADUATED MECHANICAL PROPERTIES

(71) Applicants: Jean-Marc Lefebvre, Villeneuve d'Ascq (FR); Roland Seguela, Irigny (FR); Caroline Frederix, Bruxelles (BE); Richard Assaker, Kain (BE)

(72) Inventors: Jean-Marc Lefebvre, Villeneuve d'Ascq (FR); Roland Seguela, Irigny (FR); Caroline Frederix, Bruxelles (BE); Richard Assaker, Kain (BE)

(73) Assignees: UNIVERSITE LILLE 1 SCIENCES ET TECHNOLOGIES, Villeneuve d'Ascq (FR); CENTRE NATIONALE DE LA RECHERCHE SCIENTIFIQUE, Paris (FR)

(21) Appl. No.: 14/114,522

(22) PCT Filed: Apr. 25, 2012
(86) PCT No.: PCT/EP2012/057559

§ 371 Date: Feb. 12, 2014

(30) Foreign Application Priority Data
Apr. 27, 2011 (FR) 1153600

(51) Int. Cl. A61F 2/44; B29C 45/00; A61L 27/16; A61F 2/30

(52) U.S. Cl. CPC A61F 2/4425; A61F 2/3094; B29C 45/0001; B29C 45/0003; A61L 27/16; A61F 2002/443; A61F 2002/30281; A61F 2002/30563; B29K 2023/065
USPC 623/17.15; 264/241

(57) ABSTRACT

An intervertebral disc prosthesis comprises a structure having a rigid upper plate, a rigid lower plate, and a core inserted between said plates, the structure being made from thermoplastic material, the core further comprising a thermoplastic monomaterial having graduated elastic and damping properties. According to one alternative, the core is composed of an elastomeric central nucleus surrounded by one or more rings which are referred to as rings having ranks j relative to the core, and which are more rigid.

The field of the invention is that of prostheses, notably that of intervertebral discs intended to replace natural discs.

Generally, each of the intervertebral discs of the vertebral column is composed of a central element called the nucleus pulposus, which is surrounded by a band of fibers called the annulus, and of two more rigid upper and lower plates, which provide the contact with the adjacent vertebrae.

The disc ensures the connection between two vertebral bodies and controls the movements of flexion, inclination and rotation of the vertebral column. The effects of time, stress or certain degenerative diseases cause this disc to degrade, resulting in compression of the disc and/or poor functioning thereof. This can lead to pathologies of various types which cause multiple pain of greater or lesser intensity and handicap the sufferer to a greater or lesser extent.

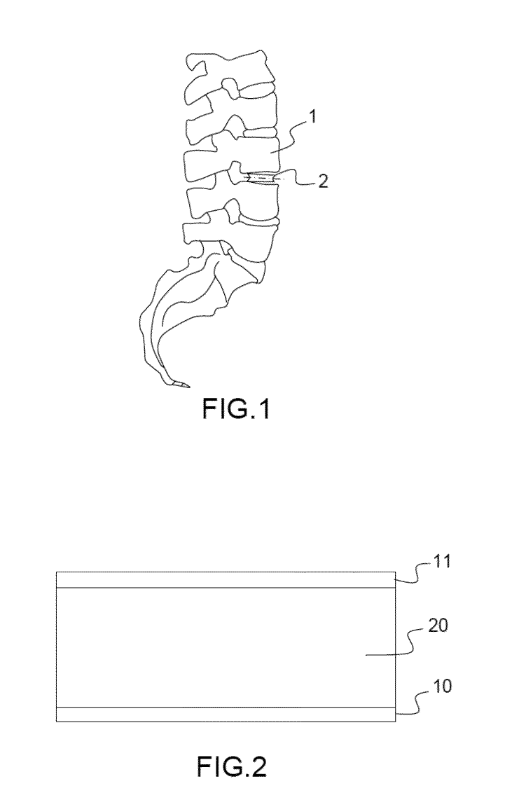

The treatment of this type of condition involves removing the diseased disc and replacing it either by an element which rigidly connects the two vertebrae concerned or by an element which is movable or deformable. FIG. 1 is a schematic view showing the positioning of a prosthesis 2 in a vertebral column 1.

Several models of prostheses have been proposed for replacing the intervertebral disc, but they are only partially satisfactory, notably in terms of their use on the cervical vertebrae.

The patent applications WO 2007 057555 and FR 2921820 have notably proposed the use of a disc comprising a rigid upper plate, a rigid lower plate, and an elastically compressible cushion placed between the two plates, the assembly being divided into two units resting one atop the other via complementary contact surfaces.

Moreover, the patent applications WO 99/30651 and U.S. Pat. No. 5,071,437 have proposed an intervertebral disc composed of an elastomeric core interposed between two rigid plates.

Furthermore, the patent application EP 0346129 A1 describes a biocompatible intervertebral disc, comprising a core placed between two plates and composed of an elastomeric nucleus surrounded by an elastomeric ring reinforced by fibers, but with the following disadvantages: presence of non-biocompatible fibers (glass, carbon), risk of loosening of said fibers, incorporation of additives (size, etc.), complexity of design.

Generally, although the known prostheses restore the intervertebral space to a value close to that provided by the healthy disc and preserve a certain degree of intervertebral mobility, they nonetheless impose particular kinematics which are specific to them. Indeed, they have their own center of rotation and plane-on-plane guides susceptible of interfering with the elements of the natural articulation (notably the articular facets). This results in a mobility that is different than the natural relative mobility of two vertebrae.

Moreover, the known prostheses do not restore the lordosis corresponding to a normal cervical or lumbar inclination.

At present, the implanted disc prostheses are composed of different parts made either entirely of metal (for example of titanium) or of a combination of metal and polymer, as is described notably in the following articles: M. J. Torrens, Cervical spondylosis; Part III: Cervical arthroplasty, Current Orthopaedics, 2005, 19, 127-134, S. Taksali, J. N. Grauer, A. R. Vaccaro, Material considerations for intervertebral disc replacement implants, The Spine Journal, 2004, 4, 231 S-238S, K. Singh, A. R. Vaccaro; T. J. Albert, Assessing the potential of total disc arthroplasty on surgeon practice patterns in North America, The Spine Journal, 4, 195S-201S. The metal plates in contact with the vertebrae surrounding the core of the prosthesis (of metal or of polymer) can be treated (roughening, and coating with hydroxyapatite or calcium phosphate) in such a way as to promote the growth of bone thereon, but they are often fixed to the vertebrae with the aid of keels or screws. However, using these fixation systems poses some risks:
weakening and rupture of the vertebra that has been drilled (screw) or hollowed out (keel) in order to insert the prosthesis;
loosening;
breaking of the screw.

Finally, the metals are much more rigid than the natural disc, and the non-resorbable biocompatible polymers presently used in the field of arthroplasty, namely polyurethane, PEEK (polyether ether ketone) and high density polyethylene, do not have a sufficient power of damping that is essential to the ability to absorb shocks. These phenomena also cause early attrition, both of the natural elements and of the prosthesis, thereby risking deterioration of the condition of the patient.

It is for this reason, in this context, that the present invention relates to an intervertebral disc prosthesis that comprises a structure having a rigid upper plate, a rigid lower plate, and a core inserted between said plates, characterized in that said structure is made from thermoplastic material, the core further comprising a thermoplastic monomaterial having graduated elastic and damping properties.

According to one alternative embodiment of the invention, the structure has a graduated thickness from one end to the other, which gives it a wedge shape, the front being thicker than the rear.

According to one alternative embodiment of the invention, the rigid upper and lower plates are composed of a mixture of polyethylenes comprising at least 50% by weight of high density polyethylene (HDPE).

According to one alternative embodiment of the invention, the rigid upper and lower plates are composed 100% of high density polyethylene (HDPE) polymer.

According to one alternative embodiment of the invention, said core is composed of an elastomeric central nucleus surrounded by one or more rings which are referred to as rings having ranks j, and which are more rigid.

According to one alternative embodiment of the invention, the central nucleus represents at least 20% of the volume of the core.

According to one alternative embodiment of the invention, the central nucleus is composed of:
50 to 100% by weight of ultra low density polyethylene (ULDPE), and
0 to 50% by weight of low density polyethylene (LLDPE).

According to one alternative embodiment of the invention, said rings are composed of:
0 to 50% by weight of ultra low density polyethylene (ULDPE);
50 to 100% by weight of low density polyethylene (LLDPE);
the ring of rank j+1 containing less ULDPE than the ring of rank j.

According to one alternative embodiment of the invention, the core comprises two rings, the ring of rank 1 being composed of a mixture comprising 50 to 75% of LLDPE, and the ring of rank 2 being composed of 75% to 100% of LLDPE.

According to one alternative embodiment of the invention, each ring is composed of two half-rings called front half-rings and of two half-rings called rear half-rings, of which the compositions are chosen in such a way that the front half-rings have a coefficient of rigidity higher than that of the rear half-rings.

According to one alternative embodiment of the invention, with the half-rings being made from mixtures of LLDPE and ULDPE, the front half-rings comprise a higher percentage of LLDPE than that of the rear half-rings.

According to one alternative embodiment of the invention, the nucleus is likewise composed of a succession of layers of elastomeric polyethylenes having different elastic and damping properties.

According to one alternative embodiment of the invention, said layers are composed of a mixture of ultra low density polyethylene (ULDPE) and low density polyethylene (LLDPE), the different layers having different percentages of LLDPE, and the layer of rank n+1 contains more LLDPE than the layer of rank n.

According to one alternative embodiment of the invention, the first layer, starting from the rear, is composed of ultra low density polyethylene (ULDPE), and the layer of rank n+1 has a percentage of LLDPE higher than that of the layer of rank n.

According to one alternative embodiment of the invention, the last layer, starting from the front, is composed of low density polyethylene (LLDPE).

According to one alternative embodiment of the invention, the intervertebral disc prosthesis comprises a central layer of ULPDE, and layers having a percentage of LLDPE increasing from said central layer.

The invention also relates to a method for producing an intervertebral disc prosthesis according to the invention, in which the structure made from thermoplastic material is obtained by co-injection molding.

According to one alternative embodiment of the method of the invention, the structure made from thermoplastic material is obtained by successive molding.

According to one alternative embodiment of the method of the invention, the structure made from thermoplastic material is obtained by thermal welding.

The invention will be better understood, and other advantages will become clear, from the following description, given as a non-limiting example, and by referring to the attached figures, in which:

FIG. 1 shows a vertebral column, and the inclusion of an artificial intervertebral disc according to the known prior art;

FIG. 2 shows a first configuration of an intervertebral disc prosthesis according to the invention;

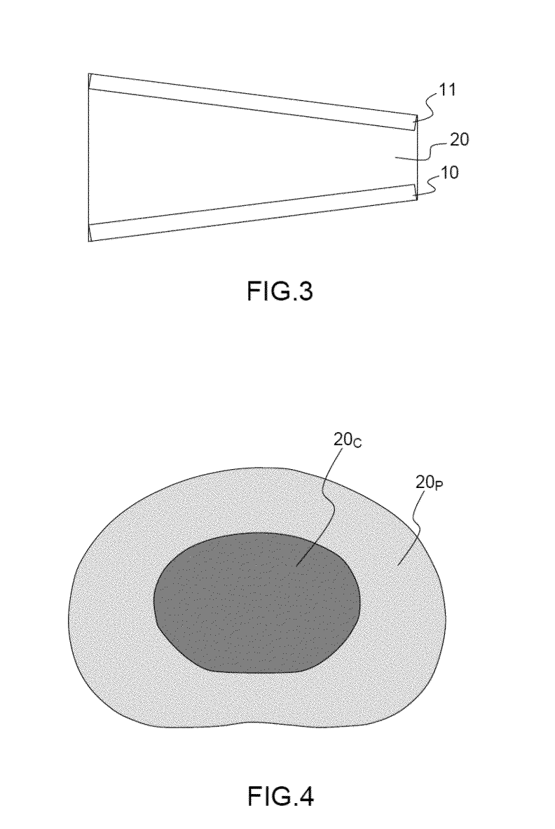

FIG. 3 shows a second configuration of an intervertebral disc prosthesis according to the invention, in what is called a wedge shape;

FIG. 4 shows a frontal cross section of a first example of a prosthesis according to the invention having a central nucleus and a peripheral ring;

FIG. 5 shows a frontal cross section of a first example of a disc prosthesis according to the invention having a central nucleus and two peripheral rings;

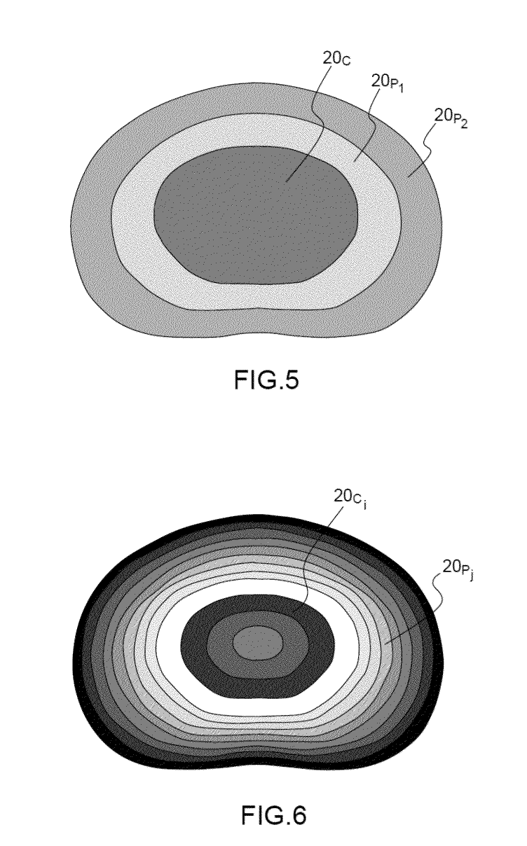

FIG. 6 shows a frontal cross section of a first example of a prosthesis according to the invention having a central nucleus and a set of peripheral rings;

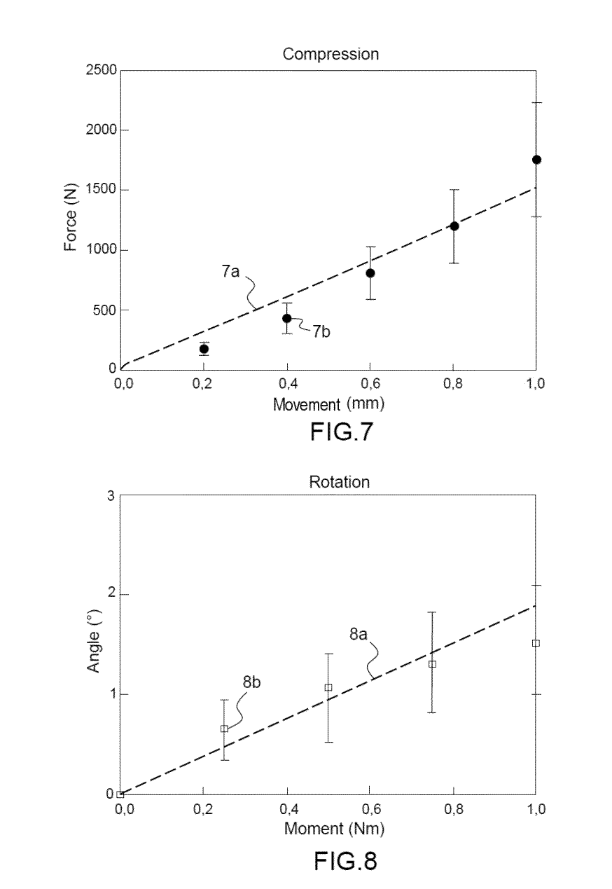

FIG. 7 shows a simulation of compression of a functional unit C5-C6 compared to experience;

FIG. 8 shows a simulation of rotation of a functional unit C5-C6 compared to experience;

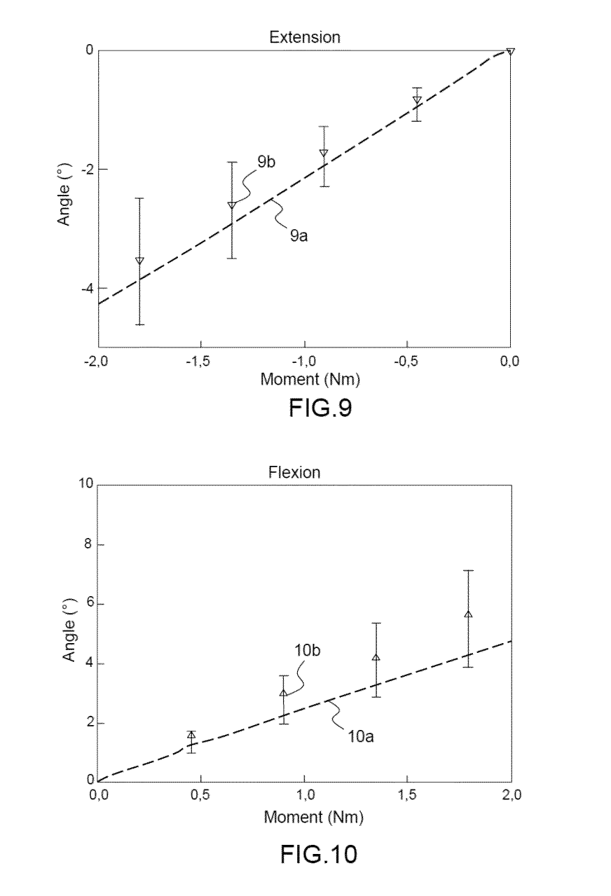

FIG. 9 shows a simulation of extension of a functional unit C5-C6 compared to experience;

FIG. 10 shows a simulation of flexion of a functional unit C5-C6 compared to experience;

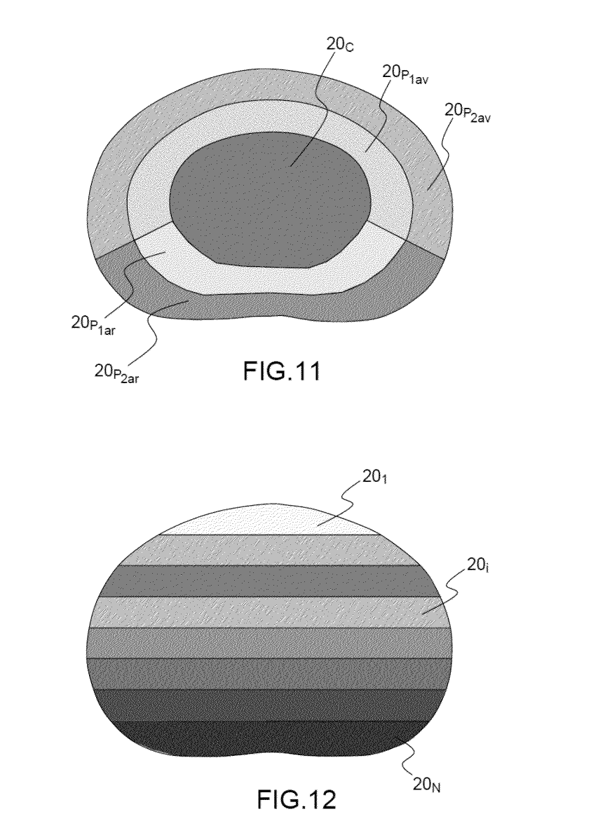

FIG. 11 shows a frontal cross section of a second example of an intervertebral disc prosthesis according to the invention;

FIG. 12 shows a frontal cross section of a third example of an intervertebral disc prosthesis according to the invention.

Generally, the intervertebral disc prosthesis according to the invention comprises, as shown in FIG. 2 which relates to a first configuration of the intervertebral disc prosthesis, a rigid lower plate 10, a rigid upper plate 11, and an elastically deformable central part 20 called the core having, at rest, the shape of a disc and having graduated mechanical properties in terms of damping for the central part and in terms of rigidity for the peripheral part, as will be explained in more detail in the description below.

In a second configuration of the invention, the prosthesis can advantageously also have a graduated thickness from one end to the other, giving said prosthesis a wedge shape, as shown in FIG. 3. This geometry, combined with the deformability of the core, can help restore the lordosis of the normal cervical or lumbar spine, which the degeneration has in general destroyed. Indeed, a prosthesis of this kind permits perfect restoration of the intervertebral space by taking into account the inclination of one vertebra to the other in the stack, which leads to the lordosis needed for the normal biomechanics of the spine as a whole.

The disc prosthesis of the present invention generally has graduated mechanical properties, notably ensuring good damping properties in the central part, and ensuring the required rigidity in the peripheral part.

In a first example of the disc prosthesis of the invention, FIG. 4 shows a frontal cross section of the core and reveals the central part 20_{C} of the core, and the peripheral part identified by a ring 20_{P}. Indeed, to be more precise, it is necessary that the core of the disc has, in its central part, a zone of lesser rigidity than the peripheral zone of said core and that it ensures good damping.

An alternative embodiment of this prosthesis is shown in FIG. 5 and has two peripheral rings 20_{P1} and 20_{P2}.

More generally, the prosthesis of the invention can have a series of rings 20_{Pj}, as is shown in the frontal cross section of such a disc prosthesis in FIG. 6, and also a sub-assembly of parts 20_{Ci} for ensuring the required graduated properties within the nucleus.

This prosthesis is therefore in the form of a component which, as regards the two adjacent vertebrae between which it is intended to be positioned, does not impose any forced connection for amplitudes of natural movement. It follows that the natural means of guiding this relative movement remain dominant (notably the posterior articular facets), and their integrity is preserved.

Thus, according to this alternative embodiment, the prosthesis of the present invention has graduated mechanical properties ranging from a flexible center to a more rigid structure at the ends, and it reproduces properties close to those of the natural discs by transmitting the stresses between the adjacent vertebrae during their movement, while ensuring good cohesion. The disc of the present invention thus allows the possibility of modulation of the rigidity to be combined with the ability for damping.

By virtue of its graduated mechanical properties, the disc prosthesis of the present invention makes it possible to vary the deformation behavior of the disc according to the load, the position and the movement of the vertebrae concerned.

The disc prosthesis can be composed of various polymers, preferably polyethylenes, referred to below by the abbreviation PEs, which have the advantage of being biocompatible, or alternatively polyurethane.

Examples of the polyethylenes that can be used are ultra low density polyethylenes (ULDPE), low density polyethylenes (LLDPE), and high density polyethylenes (HDPE). The table below lists some of the mechanical properties of these polymers:

| Properties | HDPE | LLDPE | ULDPE |
|---|---|---|---|
| Density (g · cm⁻³) | 0.94-0.95 | 0.9-0.94 | 0.86-0.9 |
| Young's modulus (Mpa) | 1050-1400 | 260-895 | <260 |
| Tensile strength (Mpa) | 22-31 | 13-45 | 17-34 |
| Yield strength (Mpa) | 18-31 | 7-19 | <7 |
| Flexural modulus (Mpa) | 1000-1550 | 275-1100 | <275 |
| Poisson's ratio | 0.45 | 0.46 | 0.47 |

It will be noted that the great variety of molecular architectures (length of chains, rate, length and distribution of the branches) is the cause of a wide diversity in the properties of polyethylenes, notably with the mechanical behavior being able to vary from a very rigid material (case of high density polyethylene, E≈1.5 GPa) to an elastomeric material (case of ultra low density polyethylene, E≈5 MPa). Moreover, the different PEs have the same chemical nature, which allows them to be combined with the aim of obtaining materials having locally controlled properties.

Advantageously, the disc prosthesis can be produced by co-injection molding or by thermal molding and/or thermal welding of the various constituent components, without using a third body such as glue or adhesive, as is explained below.

Indeed, when parts made of PEs in the molten state (or molten at the surface) are placed in contact, it is possible to obtain an interdiffusion of the macromolecular chains at the interface of the constituent elements of different PEs, so as to preserve a perfect coherence of the assembly from the chemical and mechanical points of view. Moreover, by mixing in the molten state, it is also possible to obtain materials of intermediate properties to the parent materials. A great advantage of these methods is that there is no need to add a third material that might not be biocompatible.

The very nature of the PEs means that a prosthesis having graduated mechanical properties can be produced through judicious assembly of the different PEs of interest, so as to approximate the mechanical behavior of the natural disc.

### FIRST EXAMPLE OF AN INTERVERTEBRAL DISC PROSTHESIS ACCORDING TO THE INVENTION

This first example of a prosthesis of the invention comprises two rigid plates 10 and 11 composed of a rigid polyethylene, for example a high density polyethylene (HDPE) or a mixture of polyethylenes comprising HDPE.

The core 20 is composed of an elastomeric central nucleus 20_{C} of variable size and of a peripheral region 20_{P}, as is illustrated in FIG. 5. More precisely, the peripheral part comprises at least one ring of a more rigid nature.

According to a preferred embodiment of the invention, the central nucleus is composed 100% of ULDPE.

The ring surrounding the elastomeric core can be a single ring composed of a mixture of ultra low density polyethylene (ULDPE) and low density polyethylene (LLDPE) with variable proportions of each component. Typically, the ring can advantageously be composed of a ULDPE/LLDPE mixture comprising at least 50% LLDPE or can be composed of pure LLDPE.

Advantageously, the core comprises a monomaterial or, in other words, a single material comprising a mixture of ultra low density polyethylene (ULDPE) and low density polyethylene (LLDPE) with variable proportions of each component, so as to obtain graduated elastic and damping properties.

The applicant has demonstrated that the disc prosthesis according to the invention is able to withstand the compression forces in a satisfactory manner and permits movements of rotation, flexion and lateral inclination of the cervical spine under normal conditions of stress.

For this purpose, the applicant used the finite element approach to create a model of a functional unit C5-C6 that corresponds to the assembly consisting of vertebra C5, prosthesis, vertebra C6 and ligaments, and that integrates the intervertebral disc prosthesis comprising a nucleus made of ULDPE constituting 20% of the volume of the core, a ring made of LLDPE, and plates made of HDPE.

The results of the compression stress shown in FIG. 7, of the rotation stress shown in FIG. 8, of the extension stress shown in FIG. 9 and of the flexion stress shown in FIG. 10 are compared against the experimental data found in the literature. The good agreement between the experimental data and the simulation demonstrates the good performance achieved by virtue of the prosthesis of the present invention. More precisely, the straight line 7a relates to the modeling results, the curved set of points 7b relating to data taken from the article by Shea et al. 1991 (Variations of stiffness and strength along the human cervical spine, Journal of Biomechanics, 24 (2): 92-107, 1991), the straight line 8a relates to the modeling results, the curved set of points 8b relating to data taken from the article by Goel and Clausen 1998 (Prediction of load sharing among spinal components of a c5-c6 motion segment using the finite element approach, Spine, 23 (6): 684-69, 1998), the straight line 9a relates to the modeling results, the curved set of points 9b relating to data taken from the article by Moroney et al. 1988 (Load-displacement properties of lower cervical spine motion segments, Journal of Biomechanics, 21 (9): 769-779, 1988), and the straight line 10a relates to the modeling results, the set of points 10b relating to data also taken from Moroney et al. 1998.

According to another embodiment, the intervertebral disc prosthesis can have an arrangement in which the properties can be graduated in the manner necessary for using the disc as an intervertebral prosthesis, for example by the succession of rings 20_{Pj} having different mechanical properties with the modulus increasing from the inside outward. Typically, the elastomeric central nucleus can represent at least 20% of the total volume of the core.

The ring surrounding the elastomeric core can comprise at least two peripheral rings, as shown in FIG. 5, or a succession of rings composed of a mixture of ultra low density polyethylene (ULDPE) and low density polyethylene (LLDPE) with variable proportions of each component, as shown in FIG. 6. Typically, the nucleus is thus surrounded by two rings, the first being composed of a mixture comprising 50 to 75% of LLDPE, while the second comprises 75 to 100% of LLDPE.

In the case where one or more rings are present as in the example shown in FIG. 6, the ring of rank j is richer in LLDPE than the ring of rank j−1, the aim of this being to maintain a property that is graduated in a manner suitable for the intended uses.

### SECOND EXAMPLE OF AN INTERVERTEBRAL DISC PROSTHESIS ACCORDING TO THE INVENTION

In this example, the intervertebral disc prosthesis comprises a structure which is similar to that of the first example and in which the properties of the ring can be differentiated between the front and rear of the disc.

The core is likewise composed of an elastomeric central nucleus of variable size surrounded by at least one ring that is more rigid, as in the first example, and surrounded by identical rigid plates.

The front half-ring can be in one piece or formed by a succession of half-rings composed of a mixture of ultra low density polyethylene (ULDPE) and low density polyethylene (LLDPE) with variable proportions of each component.

The rear half-ring can be in one piece or formed by a succession of half-rings composed of a mixture of ultra low density polyethylene (ULDPE) and low density polyethylene (LLDPE) with variable proportions of each component.

For example, when the rings are configured in the manner shown in FIG. 10, the disc has two front half-rings 20_{P1av}, 20_{P2av} and two rear half-rings 20_{P1ar}, 20_{P2ar}. To observe the lordosis (in the cervical or lumbar spine), the front half-rings can advantageously be more rigid that the rear half-rings. To do this, the rear half-ring is less rich in LLDPE than the front half-ring.

### THIRD EXAMPLE OF AN INTERVERTEBRAL DISC PROSTHESIS ACCORDING TO THE INVENTION

In this example, the graduated properties of the core of the intervertebral disc prosthesis are obtained by arranging a succession of layers 20, of PEs of different properties in stacked configuration, as shown in FIG. 12.

The first layer 20₁, starting from the front, is composed of pure LLDPE, and the following layers, from front to rear, contain a gradually increasing proportion of ULDPE, obtained by mixing ULDPE and LLDPE. This is because the flexion (to the front) of the cervical spine is less than the extension (to the rear), and so the last layer on the rear face, being the layer 20_{N}, has a lower percentage of LLDPE.

A second possibility is one in which the layers contain an increasing proportion of LLDPE starting from the central layer, which contains pure ULDPE.

A variation of the second possibility is one in which the rigidity is differentiated between front and rear by introducing a larger proportion of ULDPE in the rear layers.

In the same way as in the preceding examples, the core can be surrounded by plates identical to those previously described.

It should be noted in general that the intervertebral disc prosthesis according to the invention can be produced using conventional techniques well known to a person skilled in the art, such as co-injection molding, duplicate molding, or else thermal welding, for example by infrared or a heating plate.

These techniques have an undeniable advantage in the present invention. They permit control of the welding parameters, interdiffusion of the molecular chains, and production of single components having properties that are graduated almost without discontinuity.

Moreover, in the case where metal fixation plates are used, the prior art indicates (e.g. the Prodisc prosthesis is composed of two plates made of metal alloy, with a center made of ultra high density polyethylene, with the stability being provided by a central keel which slides in a groove prepared in the body of the vertebra) that it is possible to secure polyethylenes to such plates.

1. An intervertebral disc prosthesis comprising: a structure having a rigid upper plate, a rigid lower plate, and a core inserted between said plates, the structure being made from thermoplastic material, the core further comprising a thermoplastic monomaterial having graduated elastic and damping properties.

2. The intervertebral disc prosthesis as claimed in claim 1, wherein the structure has a graduated thickness from one end to the other, which gives it a wedge shape, the front being thicker than the rear.

3. The intervertebral disc prosthesis as claimed in claim 1, wherein the rigid upper and lower plates are composed of a mixture of polyethylenes comprising at least 50% by weight of high density polyethylene (HDPE).

4. The intervertebral disc prosthesis as claimed in claim 1 wherein the rigid upper and lower plates are composed 100% of high density polyethylene (HDPE) polymer.

5. The intervertebral disc prosthesis as claimed in claim 1, wherein said core is composed of an elastomeric central nucleus surrounded by one or more rings which are rings having ranks j relative to the core, and which are more rigid.

6. The intervertebral disc prosthesis as claimed in claim 1, wherein the central nucleus represents at least 20% of the volume of the core.

7. The intervertebral disc prosthesis as claimed in claim 1, wherein the central nucleus is composed of:
50 to 100% by weight of ultra low density polyethylene (ULDPE), and
0 to 50% by weight of low density polyethylene (LLDPE).

8. The intervertebral disc prosthesis as claimed in claim 1, wherein said rings are composed of:
0 to 50% by weight of ultra low density polyethylene (ULDPE), and
50 to 100% by weight of low density polyethylene (LLDPE),
and the ring of rank j+1 contains less ULDPE than the ring of rank j.

9. The intervertebral disc prosthesis as claimed in claim 1, wherein the core comprises two rings, the ring of rank 1 being composed of a mixture comprising 50 to 75% of LLDPE, and the ring of rank 2 being composed of 75% to 100% of LLDPE.

10. The intervertebral disc prosthesis as claimed in claim 8, wherein each ring is composed of at least two half-rings called front half-rings and of two half-rings called rear half-rings, of which the compositions are chosen in such a way that the front half-rings have a coefficient of rigidity higher than that of the rear half-rings.

11. The intervertebral disc prosthesis as claimed in claim 10, wherein, with the half-rings being made from mixtures of LLDPE and ULPDE, the front half-rings comprise a higher percentage of LLDPE than that of the rear half-rings.

12. The intervertebral disc prosthesis as claimed in claim 1, wherein the core is composed of a succession of layers of polyethylenes having different elastic and damping properties.

13. The intervertebral disc prosthesis as claimed in claim 12, wherein said layers are composed of a mixture of ultra low density polyethylene (ULDPE) and low density polyethylene (LLDPE), the different layers having different percentages of LLDPE, and in that the layer of rank n−1 contains more LLDPE than the layer of rank n.

14. The intervertebral disc prosthesis as claimed in claim 13, wherein the first layer, starting from the rear, is composed of ultra low density polyethylene (ULDPE), and in that the layer of rank n+1 has a percentage of LLDPE higher than that of the layer of rank n.

15. The intervertebral disc prosthesis as claimed in claim 13, wherein the last layer, starting from the front, is composed of low density polyethylene (LLDPE).

16. The intervertebral disc prosthesis as claimed in claim 12, further comprising a central layer of ULPDE, and layers having a percentage of LLDPE increasing from said central layer.

17. A method for producing an intervertebral disc prosthesis as claimed in claim 1, wherein the structure made from thermoplastic material is obtained by co-injection molding.

18. A method for producing an intervertebral disc prosthesis as claimed in claim 1, wherein the structure made from thermoplastic material is obtained by successive molding.

19. A method for producing an intervertebral disc prosthesis as claimed in claim 1, wherein the structure made from thermoplastic material is obtained by thermal welding.
